(19) United States
(12) Patent Application Publication
(10) Pub. No.: US 2008/0028542 A1
(43) Pub. Date: Feb. 7, 2008

(54) New Reactive Dyes

(75) Inventors: Warren J. Ebenezer, Greater Manchester (GB); John A. Taylor, Manchester (GB); Adrian J. Murgatroyd, Cheshire (GB); Werner Hubert Russ, Florsheim-Wicker (DE)

Correspondence Address: CONNOLLY BOVE LODGE & HUTZ, LLP, P O BOX 2207, WILMINGTON, DE, 19899, US

(73) Assignee: DyStar Textilfarben GmbH & Co. Deutschland KG, Frankfurt am Main (DE)

(21) Appl. No.: 11/597,112

(22) PCT Filed: May 11, 2005
(86) PCT No.: PCT/EP05/52148

§ 371 Date: Dec. 21, 2006

(30) Foreign Application Priority Data
May 24, 2004 (GB) 0411585.3

(51) Int. Cl. C09B 62/02; C07D 403/14; C09B 62/04; C09B 62/08

(52) U.S. Cl. 8/547; 544/209

(57) ABSTRACT

The present invention refers to dyestuffs of the formula (I) wherein A is carbon linked moieties especially chromophores; Y is N or C X¹ is halogen or tertiary ammonium, especially pyridinium; L¹ is a carbon based linking unit, optionally interrupted by O-atoms or optionally substituted N-atoms, especially ethylene or propylene R⁰, R¹, R² and R³ are independently hydrogen, C₁ to C₄ alkyl groups optionally substituted, or R² is a group of the general formula (a*), or R³ independently is a group of the general formula (b*): (a*), (b*) wherein A¹ and A² have one of the meanings of A. Y¹ and Y² have independently one of the meanings of Y X² and X³ have independently one of the meanings of X¹, R⁴, R⁵, R⁶ and R⁷ are independently hydrogen or C₁ to C₄ alkyl optionally substituted. L² and L³ are carbon based linking units, optionally interrupted by O-atoms or optionally substituted N-atoms, especially ethylene or propylene. R¹ and R² may be linked together such as to form a cyclic structure together with —N-L¹-N⁺. If R² is a group of formula (a*) or (b*) then R¹ and R³ may be linked together such as to form a cyclic structure with —N-L¹-N⁺, or R⁶ and R³ may be linked together such as to form a cyclic structure with —N⁺-L³-N—, or R⁴ and R³ may be linked together such as to form a cyclic structure with —N⁺-L²-N—, but not simultaneously, processes for the preparation of said dyesstuffs and their use for dyeing and printing hydroxy- and/or carboxamido-containing fiber materials.

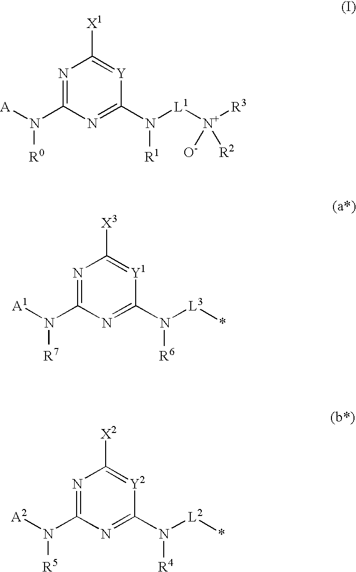

The present invention relates to the field of fibre-reactive dyes.

Good chlorine fastness is an increasingly important criterion for reactive dyes.

The inventors of the present invention have surprisingly found that dyestuffs according to the general formula (I) show high light fastness, high perspiration light fastness, good build-up properties and good solubility in salt solution and especially very good chlorine fastness properties.

The present invention claims dyestuffs or dyestuff mixtures comprising one or more dyestuffs of the formula (I) 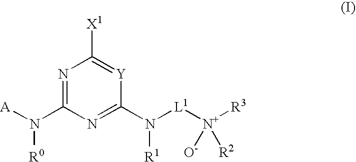
wherein
A is carbon linked moieties especially chromophores;
Y is N or C
X¹ is halogen or tertiary ammonium, especially pyridinium;
L¹ is a carbon based linking unit, optionally interrupted by O-atoms or optionally substituted N-atoms, especially ethylene or propylene
R⁰, R¹, R2 and R³ are independently hydrogen, C₁ to C₄ alkyl groups optionally substituted, or R² is a group of the general formula (a*), or R³ independently is a group of the general formula (b*) 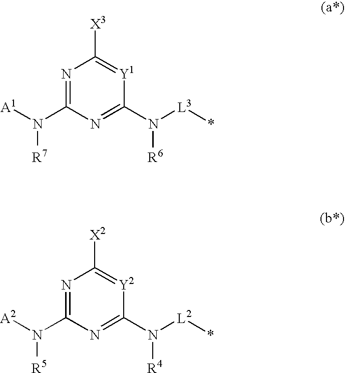
   wherein
   A¹ and A² have one of the meanings of A
   Y¹ and Y² have independently one of the meanings of Y
   X² and X³ have independently one of the meanings of X¹
   R⁴, R⁵, R⁶ and R⁷, are independently hydrogen or C₁ to C₄ alkyl optionally substituted
   L² and L³ are carbon based linking units, optionally interrupted by O-atoms or optionally substituted N-atoms, especially ethylene or propylene.
   R¹ and R² may be linked together such as to form a cyclic structure together with —N-L¹-N⁺—. If R² is a group of formula (a*) then R¹ and R³ may be linked together such as to form a cyclic structure with —N-L¹-N⁺—, or R⁶ and R³ may be linked together such as to form a cyclic structure with —N⁺-L³-N—,but not simultaneously.

R⁰, R¹, R4, R⁵, R⁶ and R⁷ may be straight-chain or branched and is for example methyl, ethyl, n-propyl, i-propyl or n-butyl. Preferably, R⁰, R¹, R4, R⁵, R⁶ and R⁷ are hydrogen or methyl, especially preferred they are hydrogen. R² is preferably hydrogen, methyl or a group of the general formula (a), R³ is preferably hydrogen, methyl or a group of the general formula (b), X¹, X² and X³ are preferred chlorine, fluorine or nicotinic acid, especially preferred chlorine. Y, Y¹ and, Y² are preferably nitrogen, L¹, L² and L³ are preferred ethylene or propylene. Each A is independently a sulphonated chromophoric system, optionally metallised, typical of those used for reactive dyes for cotton such as the radical of a mono- or disazo dye; the radical of a heavy metal complex azo dye, such as a 1:2 chromium complex, 1:2 cobalt complex and, in particular, an o,o′-1:1 copper complex monoazo or disazo dye; the radical of an anthraquinone or copper formazan dye, a nickel, copper or aluminum phthalocyanine dye or a triphendioxazine dyer especially preferred azo based chromophoric systems.

An especially preferred embodiment of the invention is a dyestuff of formula (I-1) 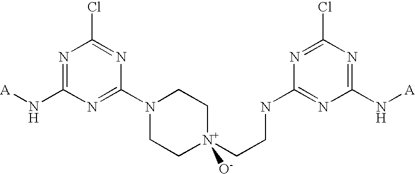
wherein A is as defined above.

The dyestuffs of the present invention can be present as a preparation in solid or liquid (dissolved) form. The dyestuff preparation an contain one ore more dyestuffs of the present inventions In solid form they generally contain the electrolyte salts customary in the case of water-soluble and in particular fibre-reactive dyes, such as sodium chloride, potassium chloride and sodium sulfate, and also the auxiliaries customary in commercial dyes, such as buffer substances capable of establishing a pH in aqueous solution between 3 and 7, such as sodium acetate, sodium borate, sodium bicarbonate, sodium citrate, sodium dihydrogen-phosphate and disodium hydrogenphosphate, small amounts of siccatives or, if they are present in liquid, aqueous solution (including the presence of thickeners of the type customary in print pastes), substances which ensure the permanence of these preparations, for example mold preventatives.

In general, the dyestuffs of the present invention are present as dye powders containing 10 to 80% by weight, based on the dye powder or preparation, of a strength-standardizing colorless diluent electrolyte salt, such as those mentioned above. These dye powders may additionally include the aforementioned buffer substances in a total amount of up to 10%, based on the dye powder. If the dyestuffs and dyestuff mixtures of the present invention are present in aqueous solution, the total dye content of these aqueous solutions is up to about 50% by weight, for example between 5 and 50% by weight, and the electrolyte salt content of these aqueous solutions will preferably be below 10% by weight, based on the aqueous solutions. The aqueous solutions (liquid preparations) may include the aforementioned buffer substances in an amount which is generally up to 10% by weight, for example 0.1 to 10% by weight, preference being given to up to 4% by weight, especially 2 to 4% by weight.

A dyestuff of the formula (I) may for example be prepared by reacting a dyestuff of the formula (II) 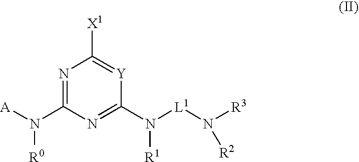
wherein
A is independently carbon linked moieties especially chromophores;
Y is independently N or C
X¹ is halogen or tertiary ammonium, especially pyridinium;
L¹ is a carbon based linking unit, optionally interrupted by O-atoms or optionally substituted N-atoms, especially ethylene or propylene
R⁰, R¹, R² and R³ are independently hydrogen, C₁ to C₄ alkyl groups optionally substituted, or R² is a group of the general formula (a*) or (b*) 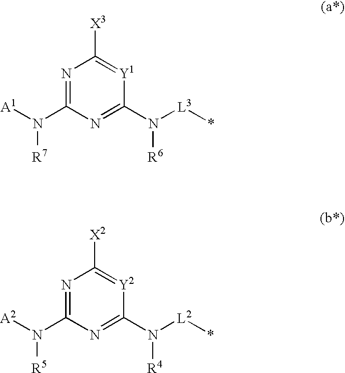
   wherein
   A¹ and A² have one of the meanings of A
   Y¹ and Y² are as defined above
   X² and X³ have one of the meanings of X¹
   R⁴, R⁵, R⁶ and R⁷, are independently hydrogen or C₁ to C₄ alkyl optionally substituted
   L² and, L³ are carbon based linking unit, optionally interrupted by O-atoms or optionally substituted N-atoms, especially ethylene or propylene
with peracetic acid in diluted acetic acid (36-40% solution), stirring for 16 hours and precipitation of the product by addition of methylated spirits. Alternatively it is possible to have hydroxyl- and/or carboxamido-containing fibre materials dyed with the inventive dyestuffs by the application of dyestuffs of the formula (II) and then oxidizing the dyed material using common oxidizing agents such as perborate together with a bleach activator such as tetraacetyletylenediamine.

The dyestuffs of the formula (II) are known and described in for example WO9905224. They can be prepared as described in the cited literature.

The dyestuffs and dyestuff mixtures of the instant invention are suitable for dyeing and printing hydroxy- and/or carboxamido-containing fibre materials by the application and fixing methods numerously described in the art for fibre-reactive dyes. They provide exceptionally bright, exceptionally strong and economic shades. Such dyes especially when used for exhaust dyeing of cellulosic materials can exhibit excellent properties including build-up, aqueous solubility, light-fastness, chlorine fastness, wash off and robustness to process variables. They are also wholly compatible with similar dyes designed for high temperature (80-100° C.) application to cellulosic textiles, and thus lead to highly reproducible application processes, with short application times.

The present invention therefore also provides for use of the inventive dyestuffs and dyestuff mixtures for dyeing and printing hydroxy- and/or carboxamido-containing fibre materials and processes for dyeing and printing such materials using a dyestuff and a dyestuff mixture, respectively, according to the invention and also for the use in digital printing techniques, especially for ink jet printing. Usually the dyestuff or dyestuff mixture is applied to the substrate in dissolved form and fixed on the fibre by the action of an alkali or by heating or both.

Hydroxy-containing materials are natural or synthetic hydroxy-containing materials, for example cellulose fiber materials, including in the form of paper, or their regenerated products and polyvinyl alcohols. Cellulose fibre materials are preferably cotton but also other natural vegetable fibres, such as linen, hemp, jute and ramie fibres. Regenerated cellulose fibres are for example staple viscose and filament viscose.

Carboxamido-containing materials are for example synthetic and natural polyamides and polyurethanes, in particular in the form of fibres, for example wool and other animal hairs, silk, leather, nylon-6,6, nylon-6, nylon-11, and nylon-4.

Application of the inventive dyestuffs and dyestuff mixtures is by generally known processes for dyeing and printing fibre materials by the known application techniques for fibre-reactive dyes. The dyestuffs and dyestuff mixtures according to the present invention are highly compatible with similar dyes designed for high temperature (80-100° C.) applications and are advantageously useful in exhaust dyeing processes.

Similarly, the conventional printing processes for cellulose fibres, which can either be carried out in single-phase, for example by printing with a print paste containing sodium bicarbonate or some other acid-binding agent and the colorant, and subsequent steaming at appropriate temperatures, or in two phases, for example by printing with a neutral or weakly acid print paste containing the colorant and subsequent fixation either by passing the printed material through a hot electrolyte-containing alkaline bath or by overpadding with an alkaline electrolyte-containing padding liquor and subsequent batching of this treated material or subsequent steaming or subsequent treatment with dry heat, produce strong prints with well defined contours and a clear white ground. Changing fixing conditions has only little effect on the outcome of the prints. Not only in dyeing but also in printing the degrees of fixation obtained with dyestuffs or dyestuff mixtures of the invention are very high. The hot air used in dry heat fixing by the customary thermofix processes has a temperature of from 120 to 200° C. In addition to the customary steam at from 101 to 103° C., it is also possible to use superheated steam and high pressure steam at up to 160° C.

Acid-binding agents responsible for fixing the dyes to cellulose fibers are for example water-soluble basic salts of alkali metals and of alkaline earth metals of inorganic or organic acids, and compounds which release alkali when hot, Of particular suitability are the alkali metal hydroxides and alkali metal salts of weak to medium inorganic or organic acids, the preferred alkali metal compounds being the sodium and potassium compounds. These acid-binding agents are for example sodium hydroxide, potassium hydroxide, sodium carbonate, sodium bicarbonate, potassium carbonate, sodium formate, sodium dihydrogenphosphate and disodium hydrogenphosphate.

Treating the dyestuffs and dyestuff mixtures according to the invention with the acid-binding agents with or without heating bonds the dyestuffs chemically to the cellulose fibers. Especially the dyeings on cellulose, after they have been given the usual aftertreatment of rinsing to remove unfixed dye portions, show excellent properties.

The dyeings of polyurethane and polyamide fibres are customarily carried out from an acid medium. The dyebath may contain for example acetic acid and/or ammonium sulfate and/or acetic acid and ammonium acetate or sodium acetate to bring it to the desired pH. To obtain a dyeing of acceptable levelness it is advisable to add customary leveling auxiliaries, for example based on a reaction product of cyanuric chloride with three times the molar amount of an aminobenzenesulfonic acid or aminonaphthalenesulfonic acid or based on a reaction product of for example stearylamine with ethylene oxide. In general the material to be dyed is introduced into the bath at a temperature of about 40° C. and agitated therein for some time, the dyebath is then adjusted to the desired weakly acid, preferably weakly acetic acid, pH, and the actual dyeing is carried out at temperature between 60 and 98° C. However, the dyeings can also be carried out at the boil or at temperatures up to 120° C. (under superatmospheric pressure).

If used in the inkjet process the inventive dyestuffs are formulated in aqueous inks, which then are sprayed in small droplets directly onto the substrate. There is a continuous process, in which the ink is pressed piezoelectrically through a nozzle at a uniform rate and deflected onto the substrate by an electric field, depending on the pattern to be produced, and there is an interrupted inkjet or drop-on-demand process, in which the ink is expelled only where a colored dot is to be placed. The latter form of the process employs either a piezoelectric crystal or a heated cannula (bubble or thermojet process) to exert pressure on the ink system and so eject an ink droplet. These techniques are described in Text. Chem. Color, volume 19 (8), pages 23 ff. and volume 21, pages 27 ff.

The printing inks for the inkjet process contain one or more inventive dyes of the formula (I) in amounts, for example, of from 0.1% by weight to 50% by weight, preferably in amounts of from 1% by weight to 30% by weight, and with particular preference in amounts of from 5% by weight to 25% by weight, based on the total weight of the ink. The pH of these printing inks is preferably adjusted to 7.0 to 9.0 by use of a suitable buffer system. This system is used in amounts of 0.1-3% by weight, preferably in 0.5-1.5% by weight, based on the total weight of the ink.

Useful buffer systems for printing inks include for example borax, disodium hydrogenphosphate, modified phosphonates, and buffer systems as described in: “Chemfe der Elemente”, VCH Verlagsgesellschaft mbH, 1^{st} edition 1988, pages 665 to 666, Holleman-Wiberg, Lehrbuch der anorganischen Chemie, WDG & Co. Verlage 47th to 56th edition, pages 109 to 110, Laborchemikalienverlag der Fa. MERCK, Darmstadt, Ausgabe 1999, pages 1128 to 1133, “Der Fischer Chemicals Katalog” (Fischer Scientific UK, 1999) pages 409 to 411, Riedel-de Haën, Laborchemikalien 1996, pages 946 to 951, Riedel-de Haën, Labor-Hilfstabellen No. 6, buffer solutions.

The dyestuffs of the formula (I) used in the inks of the inkjet process have in particular a salt content of less than 0.1% by weight, for example 0.01 to 0.099% by weight, based on the weight of the dyes. If necessary, the dyes have to be desalted, for example by membrane separation processes, before use in the inks according to the invention. For use of inks in the continuous flow process, a conductivity of 0.5 to 25 mS/m can be set by adding an electrolyte. Useful electrolytes include for example lithium nitrate and potassium nitrate.

The inks for the inkjet process may include further organic solvents with a total content of 1-20%, preferably 1-15% by weight, based on the total ink weight. Suitable organic solvents include for example alcohols, eg. methanol, ethanol, 1-propanol, isopropanol, 1-butanol, tert-butanol and pentyl alcohol; polyhydric alcohols, eg. 1,2-ethanediol, 1,2,3-propanetriol, butanediol, 1,3-butanediol, 1,4-butanediol, 1,2-propanediol, 2,3-propanediol, pentanediol, 1,4-pentanediol, 1,5-pentanediol, 1,2-hexanediol, D,L-1₁2-hexanediol, 1,6-hexanedfol, and 1,2-octanediol; polyalkylene glycols, eg. polyethylene glycol, polypropylene glycol; alkylene glycols having 2 to 8 alkylene groups, eg. monoethylene glycol, diethylene glycol, triethylene glycol, tetraethylene glycol, thioglycol, thiodiglycol, butyltriglycol, hexylene glycol, propylene glycol, dipropylene glycol, tripropylene glycol; low alkyl ethers of polyhydric alcohols, eg. ethylene glycol monomethyl ether, ethylene glycol monoethyl ether, ethylene glycol monobutyl ether, diethylene glycol monomethyl ether, diethylene glycol monoethyl ether, diethylene glycol monobutyl ether, diethylene glycol monohexyl ether, triethylene glycol monomethyl ether, triethylene glycol monobutyl ether, tripropylene glycol monomethyl ether, tetraethylene glycol monomethyl ether, tetraethylene glycol monobutyl ether, tetraethylene glycol dimethyl ether, propylene glycol monomethyl ether, propylene glycol monoethyl ether, propylene glycol monobutyl ether and tripropylene glycol isopropyl ether; polyalkylene glycol ethers, eg. polyethylene glycol monomethyl ether, polypropylene glycol glycerol ether, polyethylene glycol tridecyl ether and polyethylene glycol nonylphenyl ether; amines, eg. methylamine, ethylamine, triethylamine, diethylamine, dimethylamine, trimethylamine, dibutylamine, diethanolamine, triethanolamine, N-acetylethanolamine, N-formylethanolamine, ethylenediamine; urea derivatives, eg. urea, thiourea, N-methylurea, N,N′-dimethylurea, ethyleneurea, 1,1,3,3-tetramethylurea; amides, eg.: dimethylformamide, dimethylacetamide and acetamide; ketones or ketoalcohols, eg. acetone and diacetone alcohol, cyclic ethers, eg. tetrahydrofuran, trimethylolethane, trimethylolpropane, 2-butoxyethanol, benzyl alcohol, 2-butoxyethanol, gamma-butyrolactone and c-caprolactam; also sulfolane, dimethylsulfolane, methylsulfolane, 2,4-dimethylsulfolane, dimethyl sulfone, butadiene sulfone, dimethyl sulfoxide, dibutyl sulfoxide, N-cyclohexylpyrrolidone, N-methyl-2-pyrrolidone, N-ethylpyrrolidone, 2-pyrrolidone, 1-(2-hydroxyethyl)-2-pyrrolidone, 1-(3-hydroxypropyl)-2-pyrrolidone, 1,3-dimethyl-2-imidazolidinone, 1,3-dimethyl-2-imldazollnone, 1,3-bismethoxymethylimidazolidine, 2-(2-methoxyethoxy)ethanol, 2-(2-ethoxyethoxy)ethanol, 2-(2-butoxyethoxy)ethanol, 2-(2-propoxyethoxy)ethanol, pyridine, pipeddine, butyrolactone, trimethylolpropane, 1,2-dimethoxypropane, dioxane, ethyl acetate, ethylenediaminetetraacetate, ethyl pentyl ether, 1,2-dimethoxypropane and trimethylolpropane.

The printing inks for the inkjet process may further include the customary additives, for example viscosity moderators to set viscosities in the range from 1.5 to 40.0 mPa*s in a temperature range from 20 to 50° C. Preferred inks have a viscosity of 1.5 to 20 mPa*s and particularly preferred inks have a viscosity of 1.5 to 15 mPa*s.

Useful viscosity moderators include rheological additives, for example: polyvinylcaprolactam, polyvinylpyrrolidone and their copolymers, polyetherpolyol, associative thickeners, polyurea, polyurethane, sodium alginates, modified galactomannans, polyetherurea, polyurethane and nonionic cellulose ethers. As further additives these inks may include surface-active substances to set surface tensions of 20 to 65 mN/m, which are adapted if necessary as a function of the process used (thermal or piezotechnology). Useful surface-active substances is include for example:nonionic surfactants, butyldiglycol, 1,2-hexanediol. The inks may further include customary additives, for example substances to inhibit fungal and bacterial growth in amounts of 0.01 to 1% by weight based on the total weight of the ink.

The inks may be prepared in a conventional manner by mixing their components in water in the desired proportions.

The Examples hereinbelow serve to illustrate the inventions Parts and percentages are by weight, unless otherwise stated. Parts by weight relate to parts by volume as the kilogram relates to the litre.

The compounds described in the Examples in terms of a formula are indicated in the form of the free acids; in general, they are prepared and isolated in the form of their alkali metal salts, such as lithium, sodium or potassium salts, and used for dyeing in the form of their salts. The starting compounds and components mentioned in the following Examples, especially Table Examples, in the form of the free acid can similarly be used in the synthesis as such or in the form of their salts, preferably alkali metal salts.

The visible region absorption maximum (λmax) reported for the dyes of the invention were determined in aqueous solution with reference to their alkali metal salts. In the Table Examples, the λmax values appear in parentheses following the reported hue; the reported wavelength is in nm.

### EXAMPLE 1

Peracetic acid in acetic acid (36-40% solution, 0.2g, 1 mmol) was added in one portion to a stirred solution of bis-monochlorotriazinyl dye (1)(2.31 g, 1 mmol) in water (100 mls) at 20° C. and the pH adjusted and maintained to 9.

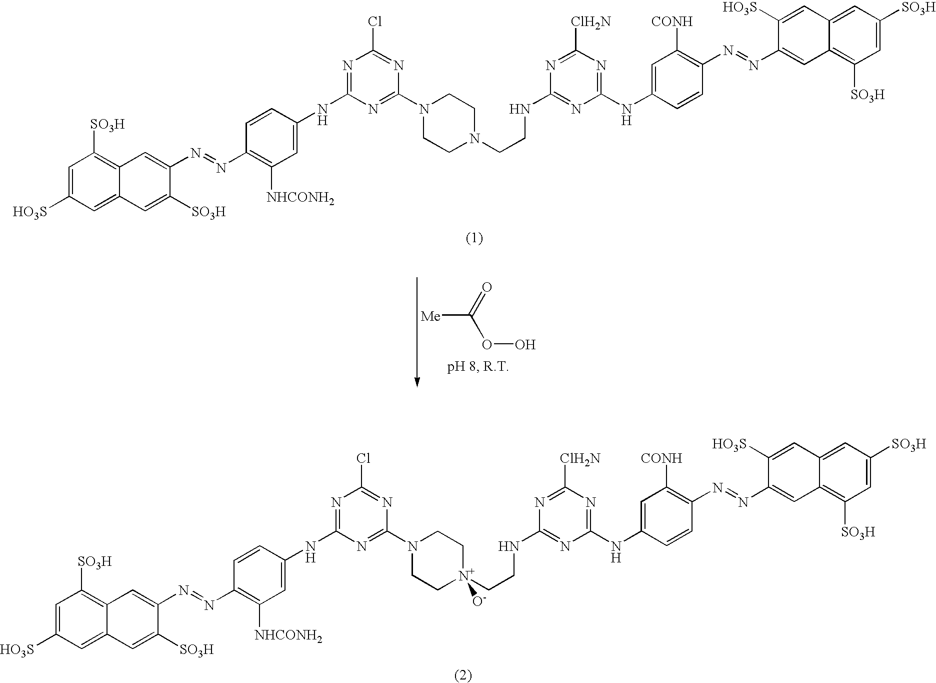

After 16 hours the reaction was complete and methylated spirits was added to precipitate the product. The resulting precipitate was filtered off and dried to give an orange powder (1 .4g, 80% yield). HPLC analysis revealed the solid to be a single component. Analytical data were in full agreement with the structure (2)
ₘₐₓ=433nm, ₘₐₓ=59000.

The structures (3)-(17), were prepared in an analogous fashion to example 1. In each case analytical data were in full agreement with the respective structures. The UV-spectra of these dyestuffs are given in table 1.

TABLE 1
UV Data

| example | structure | max/nm | max |
|---|---|---|---|
| 2 | 3 | 517 | 60250 |
| 3 | 4 | 520 | 78100 |
| 4 | 5 | 517 | unavailable |
| 5 | 6 | 521 | 94750 |
| 6 | 7 | 543 | 77400 |
| 7 | 8 | 477 | 80600 |
| 8 | 9 | 543 | 42300 |
| 9 | 10 | 534 | 36300 |
| 10 | 11 | 543 | 41100 |
| 11 | 12 | 549 | 38000 |
| 12 | 13 | 610 | 93400 |
| 13 | 14 | 605 | 87100 |
| 14 | 15 | 622 | 44500 |
| 15 | 16 | 480 | 63060 |
| 16 | 17 | 510 | 59000 |

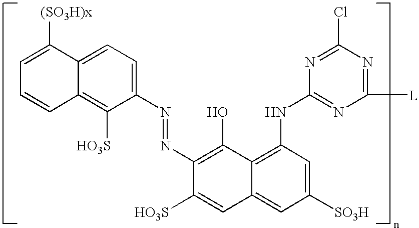

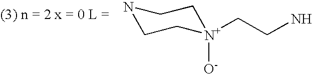

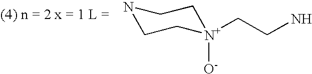

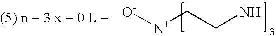

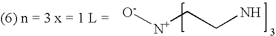

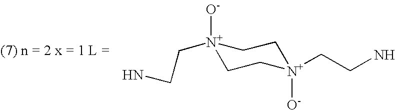

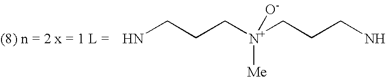

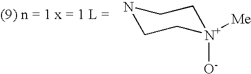

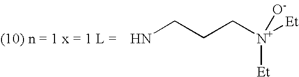

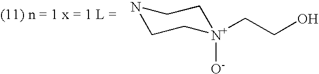

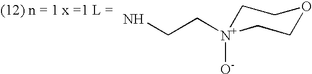

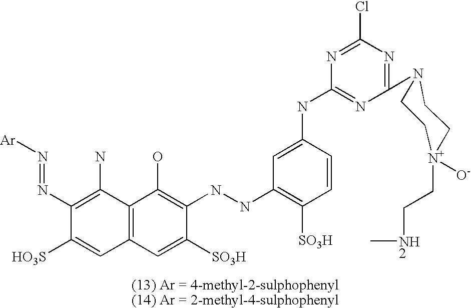

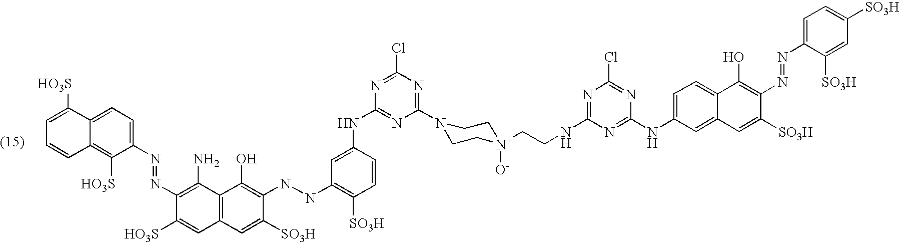

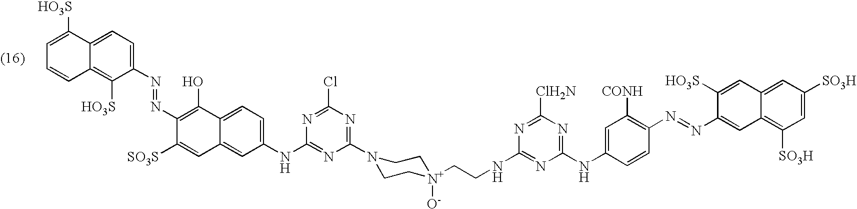

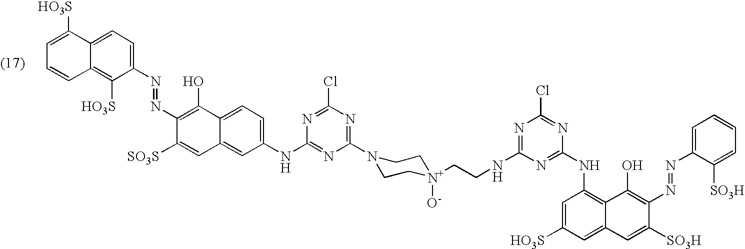

The examples (17)-(35), were prepared in an analogous fashion to example 1. In each case analytical data were in full agreement with the respective structures.

| | |
|---|---|
| | general formula |
| 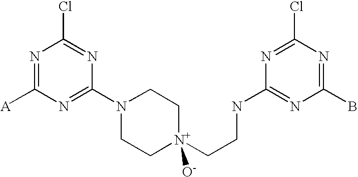 | |

A and B can have the meaning of a to p

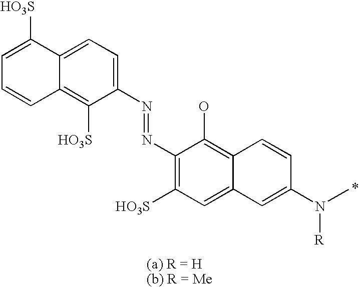

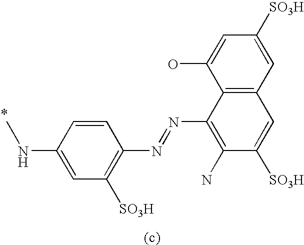

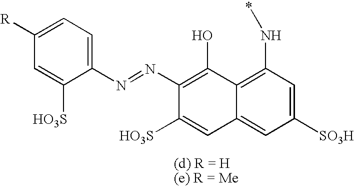

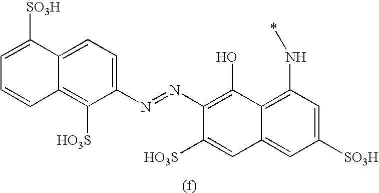

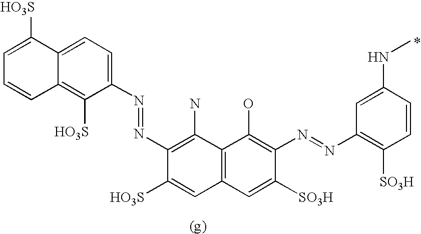

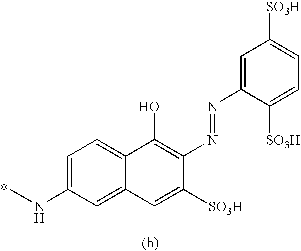

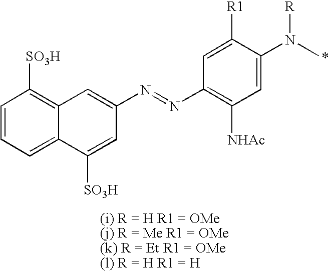

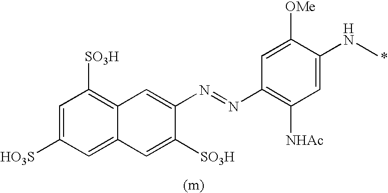

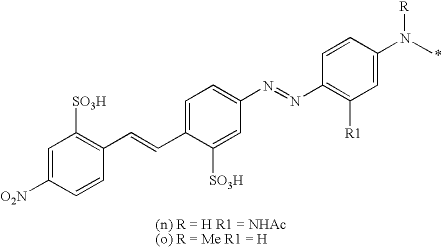

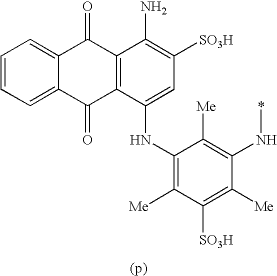

| example | A | B | max/nm | max |
|---|---|---|---|---|
| 17 | g | g | 614 | 110200 |
| 18 | d | d | 504 | 55700 |
| 19 | e | e | 510 | 53200 |
| 20 | m | m | 454 | 41900 |
| 21 | b | b | 492 | 70000 |
| 22 | f | c | 522 | 67000 |
| 23 | p | p | 589 | 24500 |
| 24 | h | h | 481 | 69000 |
| 25 | i | i | 433 | 44700 |
| 26 | j | j | 347 | 35600 |
| 27 | n | n | 420 | 87300 |
| 28 | o | o | 391 | 92100 |
| 29 | k | k | 343 | 30200 |
| 30 | l | l | 402 | 58000 |
| 31 | a | i | 484 | 55800 |
| 32 | a | m | 485 | 54700 |
| 33 | d | c | 518 | 49000 |
| 34 | c | c | 527 | 51100 |
| 35 | a | c | 509 | 64500 |

1-8. (canceled)

9. A dyestuff of the formula (I) 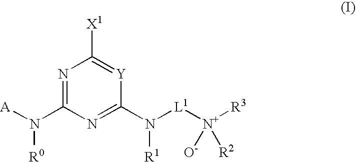
wherein
A is carbon linked moieties;
Y is N or C;
X¹ is halogen or tertiary ammonium;
L¹ is a carbon based linking unit, optionally interrupted by O-atoms or optionally substituted N-atoms;
R⁰, R¹, R² and R³ are independently hydrogen, C₁ to C₄ alkyl groups optionally substituted, or R² is a group of the general formula (a*), or R³ independently is a group of the general formula (b*) 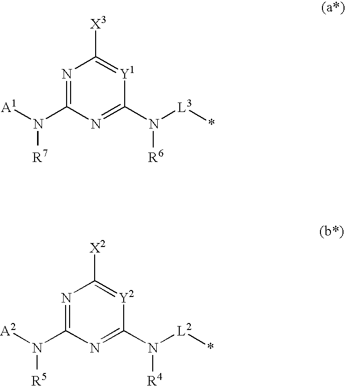
wherein
A¹ and A² have one of the meanings of A;
Y¹ and Y² have independently one of the meanings of Y;
X² and X3 have independently one of the meanings of X¹;
R⁴, R⁵, R⁶ and R⁷ are independently hydrogen or C₁ to C₄ alkyl optionally substituted;
L² and L³ are carbon based linking units, optionally interrupted by O-atoms or optionally substituted N-atoms;
R¹ and R² may be linked together such as to form a cyclic structure together with —N-L¹-N⁺—, if R² is a group of formula (a*) R¹ and R³ are optionally linked together to form a cyclic structure with —N-L -N—, or R⁶ and R³ are optionally linked together to form a cyclic structure with —N⁺-L³-N— but not simultaneously.

10. The dyestuff according to claim 9, wherein
A is a chromophore moiety;
X¹ is a halogen or pyridinium;
L¹ is an ethylene or propylene linking unit, optionally interrupted by O-atoms or optionally substituted N-atoms, and
L²and L³ are is an ethylene or propylene linking unit, optionally interrupted by O-atoms or optionally substituted N-atoms.

11. The dyestuff according to claim 9, wherein
R⁰, R¹, R⁴, R⁵, R⁶ and R⁷ are hydrogen or methyl.
R² is hydrogen or methyl or a group of the general formula (a*),
R³ is hydrogen, methyl or a group of the general formula (b*),
X¹, X² and X³ are chlorine, fluorine or nicotinic acid,
Y, Y¹ and Y² are nitrogen,
L¹, L² and L³ are ethylene or propylene and M is H, sodium or potassium.

12. A process for preparing a dyestuff of formula (I) as claimed in 9, which comprises reacting a dyestuff of the formula (II) 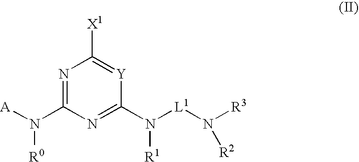
wherein
A is independently carbon linked moieties;
Y is N or C;
X¹ is halogen or tertiary ammonium;
L¹ is a carbon based linking unit, optionally interrupted by O-atoms or optionally substituted N-atoms;
R⁰, R¹, R² and R³ are independently hydrogen, C₁ to C₄ alkyl groups optionally substituted, or R² is a group of the general formula (a*), or R³ independently is a group of the general formula (b*) 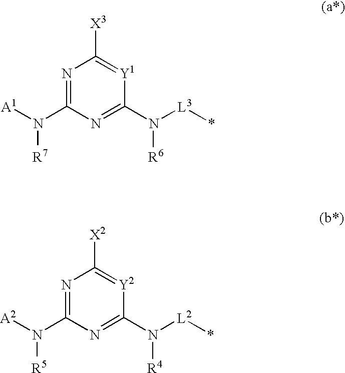
wherein
A¹ and A² have one of the meanings of A;
Y¹ and Y² have independently one of the meanings of Y;
X² and X³ have independently one of the meanings of X¹;
R⁴, R⁵, R⁶ and R⁷ are independently hydrogen or C₁ to C₄ alkyl optionally substituted;
L² and L³ are carbon based linking units, optionally interrupted by O-atoms or optionally substituted N-atoms;
R¹ and R² may be linked together such as to form a cyclic structure together with —N-L¹-N⁺—, if R² is a group of formula (a*) R² and R³ are optionally linked together to form a cyclic structure with —N-L¹-N⁺—, or R⁶ and R³ are optionally linked together to form a cyclic structure with —N⁺-L³-N— but not simultaneously with peracetic acid in diluted acetic acid (36-40% solution), stirring for 16 hours is to form a product and precipitating the product with methylated spirits.

13. A process for dyeing and printing hydroxy- and/or carboxamido-containing fibre material which comprises applying the dyestuff of the formula (I) according to claim 9 to the fibre material.

14. The process as claimed in claim 13, wherein the process uses digital printing techniques,

15. The process as claimed in claim 15, wherein the digital printing techniques are ink jet printing techniques.

16. A dyestuff preparation comprising one or more dyestuffs of the formula (1) according to claim 9 for dyeing and printing hydroxyl- and/or carboxamidocontaining fibre materials.

17. Printing inks for the inkjet process comprising one or more dyes of the formula (I) according to claim 9.

18. Hydroxy- and/or carboxamido containing fibre materials having fixed dyestuffs of the formula (I) according to claim 9.
